(19) United States
(12) Patent Application Publication
(10) Pub. No.: US 2024/0408048 A1
(43) Pub. Date: Dec. 12, 2024

(54) PET FOOD COMPOSITIONS

(71) Applicant: Hill's Pet Nutrition, Inc., Overland Park, KS (US)

(72) Inventors: Dayakar BADRI, Lawrence, KS (US); Kiran Panickar, Lawrence, KS (US); Selena Tavener, Topeka, KS (US)

(73) Assignee: Hill's Pet Nutrition, Inc., Overland Park, KS (US)

(21) Appl. No.: 18/740,073

(22) Filed: Jun. 11, 2024

Related U.S. Application Data
(60) Provisional application No. 63/507,603, filed on Jun. 12, 2023.

(51) Int. Cl. A61K 31/352; A23K 10/20; A23K 10/38; A23K 20/105; A23K 20/147; A23K 20/158; A23K 50/42; A61P 17/04

(52) U.S. Cl. CPC A61K 31/352; A23K 10/20; A23K 10/38; A23K 20/105; A23K 20/147; A23K 20/158; A23K 50/42; A61P 17/04

(57) ABSTRACT

Pet food compositions and methods for alleviating pruritus, chronic itch, erythema, alopecia, or skin and ear secretion are disclosed. In accordance with an aspect, provided is a pet food composition having from about 2 μg/g or more of quercetin; a fiber component comprising about 8 wt. % or more of a dietary fiber; and a protein, wherein all weight percentages are based on the total weight of the pet food composition on a dry matter basis. In accordance with another aspect, provided is a pet food composition having from about 2 μg/g or more of quercetin; from about 8 to about 25 wt. % of a fiber component comprising a plurality of dietary fiber, the plurality of dietary fiber comprising a soluble fiber and an insoluble fiber, wherein the soluble fiber and the insoluble fiber are present in a weight ratio of about 1:1 to about 1:15; and a protein.

### CROSS-REFERENCE TO RELATED APPLICATION

This application claims the benefit of priority from U.S. Provisional Application No. 63/507,603, filed Jun. 12, 2023, the contents of which are hereby incorporated herein by reference in their entirety.

### BACKGROUND

Atopic dermatitis (AD) is a common type of chronic inflammatory skin disease found in pets. Canines particularly suffer from atopic dermatitis, which is one of the primary reasons for a veterinary visit. Typically, atopic dermatitis is characterized by pruritus (itchiness), erythema (redness), skin and ear secretions and alopecia (hair loss). Thus, atopic dermatitis is considered to significantly reduce the quality of life for pets suffering from atopic dermatitis.

In addition to a reduced quality of life, pets suffering from atopic dermatitis often suffer from pruritic skin disorder and/or skin membrane barrier dysfunction. Skin barrier plays a critical role in preventing the entry of allergens and microorganisms into the body. The physical skin barrier is localized in the uppermost area of the epidermis called stratum corneum. Further, the epidermis is continuously regenerated by terminally differentiating keratinocytes, a process called keratinization or cornification. Cornification begins with the migration of keratinocytes from the basal to upper layers during which keratinocytes also produce lipids and extrude them into the extracellular space to form extra-cellular enriched layers to maintain skin membrane barrier function. In addition, the presence of cytokines within the skin influences various processes of keratinocyte proliferation, differentiation and cornification.

It is believed that a combination of genetic, environmental, and immunological factors influence the pathogenesis of atopic dermatitis as well as the extent of the harm from such atopic dermatitis.

There is a present need for treatments, compositions, and methods for alleviating pets suffering from atopic dermatitis and the effects thereof.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed pet food composition as well as methods for alleviating pruritus, erythema, alopecia, or skin and ear secretion. For instance, certain aspects of the invention are directed to pet food compositions and methods for alleviating pruritus and chronic itch. In accordance with an aspect of the invention, provided is a pet food composition having from about 2 μg/g or more of quercetin; a fiber component comprising about 8 wt. % or more of a dietary fiber; and a protein, wherein all weight percentages are based on the total weight of the pet food composition on a dry matter basis.

In accordance with another aspect of the invention, provided is a pet food composition having from about 2 μg/g or more of quercetin; from about 8 to about 25 wt. % of a fiber component comprising a plurality of dietary fiber, the plurality of dietary fiber comprising a soluble fiber and an insoluble fiber, wherein the soluble fiber and the insoluble fiber are present in a weight ratio of about 1:1 to about 1:15; and a protein.

A list of certain, non-limiting embodiments in accordance with aspects of the invention are provided below:

In accordance with embodiment 1, provided is a pet food composition comprising:
from about 2 μg/g or more of quercetin;
a fiber component comprising about 8 wt. % or more of a dietary fiber;
a protein; and
a fat,
wherein all weight percentages are based on the total weight of the pet food composition on a dry matter basis.

In accordance with embodiment 2, provided is a pet food composition according to embodiment 1, wherein the dietary fiber comprises a soluble fiber and an insoluble fiber.

In accordance with embodiment 3, provided is a pet food composition according to embodiment 2, wherein the pet food composition comprises a weight ratio of soluble fiber to insoluble fiber of about 1:1 to about 1:15.

In accordance with embodiment 4, provided is a pet food composition according to embodiment 3, wherein the weight ratio of soluble fiber to insoluble fiber is from about 1:3 to about 1:15.

In accordance with embodiment 5, provided is a pet food composition according to any of embodiment 2 to embodiment 4, wherein the soluble fiber is present in an amount from about 0.1 to about 5 wt. %.

In accordance with embodiment 6, provided is a pet food composition according to any of embodiment 2 to embodiment 5, wherein the insoluble fiber is present in an amount from about 1 to about 14 wt. %.

In accordance with embodiment 7, provided is a pet food composition according to any foregoing embodiment, wherein the protein is present in an amount from about 10 to about 40 wt. %.

In accordance with embodiment 8, provided is a pet food composition according to any foregoing embodiment, wherein the fat is present in an amount from about 9 to about 30 wt. % of fat.

In accordance with embodiment 9, provided is a pet food composition according to any foregoing embodiment further comprising:
from about 0.1 to about 10 wt. % of a fatty acid.

In accordance with embodiment 10, provided is a pet food composition according to embodiment 9, wherein the fatty acid comprises a polyunsaturated fatty acid.

In accordance with embodiment 11, provided is a pet food composition according to embodiment 10, wherein the polyunsaturated fatty acid comprises an omega-3 fatty acid, an omega-6 fatty acid, or a combination of two or more thereof.

In accordance with embodiment 12, provided is a pet food composition according to embodiment 11, wherein polyunsaturated fatty acid comprises an omega-3 fatty acid selected from linolenic acid, stearidonic acid, eicosatetraenoic acid, eicosapentaenoic acid, docosapentaenoic acid, docosahexaenoic acid, and a combination of two or more thereof.

In accordance with embodiment 13, provided is a pet food composition according to embodiment 11 or embodiment 12, wherein the polyunsaturated fatty acid comprises an omega-6fatty acid selected from linolenic acid, calendic acid, eicosadienoic acid, arachidonic acid, docosadienoic acid, adrenic acid, osbond acid, tetracosatetraenoic acid, tetracosapentaenoic acid, and a combination of two or more thereof.

In accordance with embodiment 14, provided is a pet food composition according to any of embodiment 11 to embodiment 13, wherein the food composition has a weight ratio of omega-6 fatty acids to omega-3 fatty acids of from about 0.5:1 to about 7:1, preferably, about 0.5:1 to about 3:1, about 0.5:1 to about 2.5:1, about 0.5:1 to about 2:1, about 0.5:1 to about 1.5:1, about 0.5:1 to about 1:1; about 1:1 to about 7:1, about 1:1 to about 3:1, about 1:1 to about 2.5:1, about 1:1 to about 2:1, about 1:1 to about 1.5:1, or about 1:1 to about 1:1; about 2:1 to about 7:1, about 2:1 to about 5:1, about 1:1 to about 4:1, about 1:1 to about 3:1, about 1:1 to about 2.5:1; about 2.5:1 to about 7:1, about 2.5:1 to about 5:1, about 2.5:1 to about 4:1, about 2.5:1 to about 3:1; about 3:1 to about 7:1, about 3:1 to about 5:1, about 3:1 to about 4:1, about 3:1 to about 3.5:1, or any range or subrange thereof.

In accordance with embodiment 15, provided is a pet food composition according to any foregoing embodiment, wherein the amount of quercetin is about 2.5 μg/g or more.

In accordance with embodiment 16, provided is a pet food composition according to any foregoing embodiment, wherein the pet food composition is free of rutin.

In accordance with embodiment 17, provided is a pet food composition comprising:
from about 2 μg/g or more of quercetin;
from about 8 to about 25 wt. % of a fiber component comprising a plurality of dietary fiber, the plurality of dietary fiber comprising a soluble fiber and an insoluble fiber, wherein the soluble fiber and the insoluble fiber are present in a weight ratio of about 1:1 to about 1:15;
a protein; and
a fat.

In accordance with embodiment 18, provided is a pet food composition according to any foregoing embodiment, wherein the pet food composition is adapted to reduce pruritus by about 25% or more, assessed about one hour after consumption of the pet food composition by a pet consuming the pet food composition once a day for six consecutive weeks.

In accordance with embodiment 19, provided is a pet food composition according to any foregoing embodiment, wherein the pet food composition is adapted to increase expression of circulating NRP1, assessed about one hour after consumption of the pet food composition by a pet consuming the pet food composition once a day for six consecutive weeks.

In accordance with embodiment 20, provided is a pet food composition according to any foregoing embodiment, wherein the pet food composition is adapted to increase expression of Bcl-2, assessed about one hour after consumption of the pet food composition by a pet consuming the pet food composition once a day for six consecutive weeks.

In accordance with embodiment 21, provided is a pet food composition according to one of embodiment 17 to embodiment 20, wherein the amount of quercetin is about 2.5 μg/g or more.

In accordance with embodiment 22, provided is a method for improving method for alleviating pruritus and/or chronic itch for a canine, the method comprising:
increasing the expression of circulating NRP1 and/or the expression of Bcl-2 by feeding the canine a pet food composition according to any foregoing embodiment.

In accordance with embodiment 23, provided is a method for improving method for alleviating pruritus and/or chronic itch for a canine, the method comprising:
adjusting the amount of circulating quercetin in a pet to about 2 μg/kg MBW or more by feeding the canine a pet food composition according to any foregoing embodiment.

In accordance with embodiment 24, provided is the method according to embodiment 23comprising increasing the circulating quercetin in the pet by about 100% or more, assessed about one hour after consumption of the pet food composition by a pet consuming the pet food composition once a day for six consecutive weeks.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a bar graph of the improvement in pruritus for canines receiving a comparative composition or a non-liming exemplary composition in accordance with aspects of the invention;

FIG. 2 is a bar graph showing the levels of NRP1 expression for canines receiving a non-limiting exemplary composition or a comparative composition in accordance with aspects of the invention;

FIG. 3 is a bar graph showing the levels of Bcl-2 expression for canines receiving an exemplary composition or a comparative composition in accordance with aspects of the invention;

FIG. 4 is a bar graph showing the levels of PTGDR2 expression for canines receiving an exemplary composition or a comparative composition in accordance with aspects of the invention;

FIG. 5 is a bar graph showing the levels of IL-27 expression for canines receiving an exemplary composition or a comparative composition in accordance with aspects of the invention; and

FIG. 6 is a bar graph showing the circulating histamine levels of atopic canines before receiving either an exemplary composition or a comparative composition and after receiving an exemplary composition or a comparative composition in accordance with aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the pet food composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as both an ancient grain and a buckwheat hull. If a particular pet food composition recites both an ancient grain and a buckwheat hull, a compound that may be characterized as both an ancient and an amaranth will serve only as either an ancient or an buckwheat hull—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (e.g., farm cats), pet cats, ferrets, birds, reptiles, rabbits, and fish.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights, and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percentage of the ingredient is based on amount of ingredient present as dry matter.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food composition as well as methods for alleviating pruritus, chronic itch, erythema, alopecia, and/or skin and ear secretion. For instance, certain preferred embodiments of the pet food compositions are adapted for alleviating pruritus and chronic itch. The inventors discovered that pet food compositions comprising a unique combination of quercetin and certain fiber components comprising particular dietary fibers/sources in amounts and ratios disclosed herein can unexpectedly reduce pruritus and chronic itch in animals, such as canines. The pet food compositions may advantageously achieve the alleviation and/or reduction of pruritus and chronic itch by effecting histamine-independent signaling pathways, such as NEFs and NRFs. Without being limited to any particular theory, certain pet food compositions may advantageously regulate the hyperinnervation of mechanically sensitive C-nerve fibers and/or reduce the outgrowth of C-nerve fibers in the epidermal region.

In accordance with an aspect of the invention, provided is a pet food composition having from about 2 μg/g or more of quercetin; a fiber component comprising about 5 wt. % or more of a dietary fiber; and a protein, wherein all weight percentages are based on the total weight of the pet food composition on a dry matter basis.

In accordance with another aspect of the invention, provided is a pet food composition having from about 2 μg/g or more of quercetin; from about 8 to about 25 wt. % of a fiber component comprising a plurality of dietary fiber, the plurality of dietary fiber comprising a soluble fiber and an insoluble fiber, wherein the soluble fiber and the insoluble fiber are present in a weight ratio of about 1:1 to about 1:15; and a protein, wherein all weight percentages are based on the total weight of the pet food composition on a dry matter basis.

The pet food compositions may be adapted to preferably reduce pruritus for a pet suffering therefrom by about 25% or more, assessed about one hour after consumption of the pet food composition by a pet consuming the pet food composition once a day for six consecutive weeks. In some embodiments, the pet food composition may be adapted to reduce pruritus for a pet suffering therefrom by about 28% or more, about 31% or more, about 33% or more, about 35% or more, about 38% or more, or about 42% or more, or about 45% or more.

Additionally or alternatively, certain pet food compositions disclosed herein may be adapted to increase expression of circulating NRP1, assessed about one hour after consumption of the pet food composition by a pet consuming the pet food composition once a day for six consecutive weeks. The pet food compositions may also be adapted to increase expression of Bcl-2, as assessed about one hour after consumption of the pet food composition by a pet consuming the pet food composition once a day for six consecutive weeks. The increase in expression of circulating NRP1 and/or increase expression of Bcl-2 may, in some embodiments, may be obtained about 24 hours after the pet consumes the pet food compositions once a day for three consecutive weeks.

The pet food compositions may achieve the above referenced reduction in pruritus, increase in expression of circulating NRP1, and/or increase in expression of Bcl-2 when the pet is provided the pet food composition in an amount according to the recommended daily nutritional intake. The recommended daily nutritional intake may be based on AAFCO and NRC nutrition recommendations; a dry matter inclusion level when daily diets are 4000 kcal/kg or less; and/or per 1000 kcal when daily diets are higher than 4000 kcal/kg in energy.

Suitable components, such as those listed below, may be included or excluded from the formulations for the pet food compositions depending on the specific combination of other ingredients and the form of the pet food compositions. In some embodiments, the pet food compositions disclosed herein may be in the form of a stand-alone pet food, as a supplement to pet food, as a pet food treat, or the like.

The pet food compositions typically include quercetin, preferably in an effective amount. The amount of quercetin in the pet food composition is generally about 2 μg/g or more, based on the total weight of the pet food composition on a dry matter basis. For example, the pet food composition may comprise quercetin in an amount from about 2 to about 35 μg/g, about 2 to about 30 μg/g, about 2 to about 25 μg/g, about 2 to about 17 μg/g, about 2 to about 15 μg/g, about 2 to about 13 μg/g; from about 2.5 to about 35 μg/g, about 2.5 to about 30 μg/g, about 2.5 to about 25 μg/g, about 2.5 to about 17 μg/g, about 2.5 to about 15 μg/g, about 2.5 to about 13 μg/g; from about 4 to about 35 μg/g, about 4 to about 30 μg/g, about 4 to about 25 μg/g, about 4 to about 17 μg/g, about 4 to about 15 μg/g, about 4 to about 13 μg/g; from about 6 to about 35 μg/g, about 6 to about 30 μg/g, about 6 to about 25 μg/g, about 6 to about 17 μg/g, about 6 to about 15 μg/g, about 6 to about 13 μg/g; about 8 to about 35 μg/g, about 8 to about 30 μg/g, about 8 to about 25 μg/g, about 8 to about 17 μg/g, about 8 to about 15 μg/g, about 8 to about 13 μg/g; from about 10 to about 35 μg/g, about 10 to about 30 μg/g, about 10 to about 25 μg/g, about 10 to about 17 μg/g, about 10 to about 15 μg/g, about 10 to about 13 μg/g; from about 12 to about 35 μg/g, about 12 to about 30 μg/g, about 12 to about 25 μg/g, about 12 to about 17 μg/g, about 12 to about 15 μg/g; from about 14 to about 35 μg/g, about 14 to about 30 μg/g, about 14 to about 25 μg/g, about 14 to about 17 μg/g; from about 17 to about 35 μg/g, about 17 to about 30 μg/g, about 17 to about 25 μg/g; from about 20 to about 35 μg/g, about 20 to about 30 μg/g, about 20 to about 25 μg/g; from about 25 to about 35 μg/g, about 25 to about 30 μg/g, or any range or subrange thereof, based on the total weight of the pet food composition on a dry matter basis. In certain cases, the pet food composition may be formulated to have about 9 μg/g or more, about 10 μg/g or more, about 11 μg/g or more, about 12 μg/g or more, about 13 μg/g or more, or about 14 μg/g or more, of quercetin, based on the total weight of the pet food composition on a dry matter basis.

The pet food compositions may, preferably, be formulated to be free of rutin or have a reduced amount of rutin, e.g., about 2 μg/g or less of rutin. In some embodiments, the pet food compositions have about 1.5 μg/g or less, about 1 μg/g or less, about 0.5 μg/g or less, or about 0.1 μg/g or less of rutin. In at least one embodiment, the pet food composition is free of rutin. The pet food compositions preferably comprise quercetin while optionally being free of rutin or having a reduced amount of rutin.

The pet food compositions typically include a fiber component comprising dietary fiber. The fiber component may comprise crude fiber in addition to the dietary fiber. The fiber component may be present in the pet food composition in an amount from about 8 to about 25 wt. %, based on the total weight of the pet food composition on a dry matter basis. In some embodiments, the pet food composition includes a fiber component in an amount from about 8 to about 23 wt. %, about 8 to about 20 wt. %, about 8 to about 16 wt. %, about 8 to about 12 wt. %, about 8 to about 10 wt. %; from about 11 to about 25 wt. %, about 11 to about 20 wt. %, about 11 to about 16 wt. %, about 11 to about 13 wt. %; from about 14 to about 25 wt. %, about 14 to about 20 wt. %, about 14 to about 16 wt. %; from about 16 to about 25 wt. %, about 16 to about 20 wt. %; from about 18 to about 25 wt. %, about 18 to about 20 wt. %, about 20 to about 25 wt. %, or any range or subrange thereof, based on the total weight of the pet food composition on a dry matter basis.

The dietary fiber is typically present in amount of about 8 wt. % or more, based on the total weight of the pet food composition on a dry matter basis. For instance, the dietary fiber may be present in the pet food compositions in an amount of about 8 wt. % or more, about 10 wt. % or more, or about 12 wt. % or more, based on the total weight of the pet food composition on a dry matter basis. In some embodiments, the pet food composition is formulated to have the dietary fiber in an amount from about 5 to about 20 wt. %, about 5 to about 16 wt. %, about 5 to about 12 wt. %, about 5 to about 10 wt. %, about 5 to about 8 wt. %, about 5 to about 7 wt. %; from about 6 to about 20 wt. %, about 6 to about 16 wt. %, about 6 to about 12 wt. %, about 6 to about 10 wt. %, about 6 to about 8 wt. %; from about 8 to about 20 wt. %, about 8 to about 16 wt. %, about 8 to about 12 wt. %, about 8 to about 10 wt. %; from about 10 to about 20 wt. %, about 10 to about 16 wt. %, about 10 to about 13 wt. %; from about 12 to about 20 wt. %, about 12 to about 16 wt. %, about 12 to about 14 wt. %; from about 14 to about 20 wt. %, or about 14 to about 16 wt. %, or any range or subrange thereof, based on the total weight of the pet food composition on a dry matter basis.

Dietary fiber refers to components of a plant which are resistant to digestion by an animal's digestive enzymes. Dietary fiber includes soluble fiber and insoluble fiber. Soluble fibers are resistant to digestion and absorption in the small intestine and undergo complete or partial fermentation in the large intestine. The soluble fibers may be from, e.g., beet pulp, guar gum, chicory root, psyllium, pectin, blueberry, cranberry, squash, apples, oats, beans, citrus, barley, or peas. Insoluble fibers can be supplied by any of a variety of sources, including, e.g., cellulose, buckwheat hull, whole-wheat products, wheat oat, corn bran, flax seed, grapes, celery, green beans, cauliflower, potato skins, fruit skins, vegetable skins, peanut hulls, and soy fiber. In at least one preferred embodiment, the pet food composition comprises buckwheat hull.

The digestible fiber may comprise soluble fiber in an amount from about 0.1 to about 5 wt. %, based on the total weight of the pet food composition on a dry matter basis. For example, the pet food composition may include soluble fiber in an amount from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the pet food composition on a dry matter basis.

The digestible fiber may, also or alternatively, comprise an insoluble fiber in an amount from about 1 to about 14 wt. %, based on the total weight of the pet food composition on a dry matter basis. In some embodiments, the amount of insoluble fiber is about 1 to about 11 wt. %, about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %; from about 3 to about 14 wt. %, about 3 to about 11 wt. %, about 3 to about 9 wt. %, about 3 to about 8 wt. %, about 3 to about 7 wt. %, about 3 to about 6 wt. %, about 3 to about 5 wt. %; from about 4 to about 14 wt. %, about 4 to about 11 wt. %, about 4 to about 9 wt. %, about 4 to about 8 wt. %, about 4 to about 7 wt. %, about 4 to about 6 wt. %; from about 5 to about 14 wt. %, about 5 to about 11 wt. %, about 5 to about 9 wt. %, about 4 to about 8 wt. %, about 5 to about 7 wt. %, about 5 to about 6 wt. %; from about 6 to about 14 wt. %, about 6 to about 11 wt. %, about 6 to about 9 wt. %, about 6 to about 8 wt. %, about 6 to about 7 wt. %; from about 8 to about 14 wt. %, about 8 to about 11 wt. %, about 8 to about 10 wt. %; from about 10 to about 14 wt. %, about 10 to about 12 wt. %, about 12 to about 14 wt. %, or any range or subrange thereof, based on the total weight of the pet food composition on a dry matter basis.

The pet food composition may preferably be adapted to have a weight ratio of soluble fiber to insoluble fiber that is from about 1:3 to about 1:15. In certain embodiments, the weight ratio of the total amount of soluble fiber to the total amount of insoluble fiber is about 1:4 to about 1:15, about 1:5 to about 1:15, about 1:6 to about 1:15, about 1:8 to about 1:15, about 1:11 to about 1:15; from about 1:3 to about 1:12, about 1:4 to about 1:12, about 1:5 to about 1:12, about 1:6 to about 1:12, about 1:8 to about 1:12, about 1:11 to about 1:12; from about 1:3 to about 1:10, about 1:4 to about 1:10, about 1:5 to about 1:10, about 1:6 to about 1:10, about 1:8 to about 1:10; from about 1:3 to about 1:8, about 1:4 to about 1:8, about 1:5 to about 1:8, about 1:6 to about 1:8; from about 1:3 to about 1:6, about 1:4 to about 1:6, about 1:5 to about 1:6; from about 1:3 to about 1:5, about 1:4 to about 1:5, including any range or subrange thereof.

Crude fiber includes indigestible components contained in cell walls and cell contents of plants, such as grains, e.g., hulls of grains such as rice, corn, buckwheat hull, and beans. The crude fiber may be present in the pet food composition in an amount from about 0.1 to about 5 wt. %, based on the total weight of the pet food composition on a dry matter basis. For example, the pet food composition may include crude fiber in an amount from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the pet food composition on a dry matter basis.

The pet food compositions typically include fat. The amount of fat in the pet food composition may be from about 9 to about 30 wt. %, based on the total weight of the pet food composition on a dry matter basis. In some embodiments, the amount of fat present in the pet food composition may be from about 9 to about 26 wt. %, about 9 to about 22 wt. %, about 9 to about 18 wt. %, about 9 to about 16 wt. %, about 9 to about 14 wt. %; from about 11 to about 30 wt. %, about 11 to about 26 wt. %, about 11 to about 22 wt. %, about 11 to about 18 wt. %, about 11 to about 16 wt. %, about 11 to about 14 wt. %; from about 12 to about 30 wt. %, about 12 to about 26 wt. %, about 12 to about 22 wt. %, about 12 to about 18 wt. %, about 12 to about 16 wt. %, or about 12 to about 14 wt. %, including ranges and subranges thereof, based on the total weight of the pet food composition on a dry matter basis.

The term “fat” generally refers to a lipid or mixture of lipids suitable for dietary consumption. The pet food compositions may include one or more fatty acid(s). The fatty acid(s) when present in the pet food composition are typically in an amount of about 0.1 to about 10 wt. %, based on the total weight of the pet food composition on a dry matter basis. For instance, the pet food composition may include one or more fatty acid(s) in a total amount from about 0.1 to about 8 wt. %, about 0.1 to about 6 wt. %, about 0.1 to about 4 wt. %, about 0.1 to about 3 wt. %, about 0.1 to about 2 wt. %; from about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 4 wt. %, about 2 to about 3 wt. %; from about 3 to about 10 wt. %, about 3 to about 8 wt. %, about 3 to about 6 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, or any range or subrange thereof, based on the total weight of the pet food composition.

The one or more fatty acid(s) may be selected from omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof. The fatty acid(s) may be a polyunsaturated fatty acid, such as an omega-3 fatty acid, an omega-6 fatty acid, or a combination of two or more thereof. Non-limiting examples of omega-3 fatty acids include those selected from linolenic acid, stearidonic acid, eicosatetraenoic acid, eicosapentaenoic acid, docosapentaenoic acid, docosahexaenoic acid, and a combination of two or more thereof. The pet food composition may include linolenic acid, eicosapentaenoic acid, docosahexaenoic acid, or a combination of two or more thereof.

Additionally or alternatively, the polyunsaturated fatty acid may comprise an omega-6 fatty acid. Examples of omega-6 fatty acids include linolenic acid, calendic acid, eicosadienoic acid, arachidonic acid, docosadienoic acid, adrenic acid, osbond acid, tetracosatetraenoic acid, tetracosapentaenoic acid, and a combination of two or more thereof. In some embodiments, the polyunsaturated fatty acid comprises an omega-6 fatty acid selected from linolenic acid, arachidonic acid, and a combination of two or more thereof.

The pet food composition, in some embodiments, comprises one or more omega-3 fatty acid and one or more omega-6 fatty acid. In some instances, the pet food composition is formulated to have a weight ratio of omega-6 fatty acid(s) to omega-3 fatty acid(s) of from about 0.5:1 to about 7:1, about 0.5:1 to about 3:1, about 0.5:1 to about 2.5:1, about 0.5:1 to about 2:1, about 0.5:1 to about 1.5:1, about 0.5:1 to about 1:1; from about 1:1 to about 7:1, about 1:1 to about 3:1, about 1:1 to about 2.5:1, about 1:1 to about 2:1, about 1:1 to about 1.5:1, or about 1:1 to about 1:1; from about 2:1 to about 7:1, about 2:1 to about 5:1, about 1:1 to about 4:1, about 1:1 to about 3:1, about 1:1 to about 2.5:1; from about 2.5:1 to about 7:1, about 2.5:1 to about 5:1, about 2.5:1 to about 4:1, about 2.5:1 to about 3:1; from about 3:1 to about 7:1, about 3:1 to about 5:1, about 3:1 to about 4:1, about 3:1 to about 3.5:1, or any range or subrange thereof.

The fat may be incorporated completely within the food composition, deposited on the outside of the pet food composition, or a mixture of the two methods (e.g., the fat may be incorporated in the pet food composition as well as deposited on the outside of the pet food composition). In some embodiments, the pet food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

Fat can be supplied by any of a variety of sources known by those skilled in the art, including meat, meat by-products, canola oil, fish oil such as anchovy oil and menhaden oil, and plants. Examples of meat fat sources include poultry fat, turkey fat, pork fat, lard, tallow, and beef fat. Examples of plant fat sources include wheat, flaxseed, rye, barley, rice, sorghum, corn, oats, millet, wheat germ, corn germ, soybeans, peanuts, cottonseed, as well as oils derived from these and other plant fat sources such as palm oil, palm kernel oil, linseed oil, canola oil, rapeseed oil, and/or olestra.

In some cases, the fat in the pet food composition is a crude fat. Crude fat may be included into the pet food compositions in the amounts disclosed above with respect of the total fat, such as from about 9 to about 30 wt. %, based on the total weight of the pet food composition on a dry matter basis. In some embodiments, the pet food composition comprises crude fat in an amount of about 11 to about 30 wt. %, about 14 to about 25 wt. %, about 14 to about 22 wt. %, based on the total weight of the pet food composition on a dry matter basis. In some cases, it may be preferable that about 50 wt. % or more, about 60 wt. % or more, about 70 wt. % or more, about 80 wt. % or more, or about 90 wt. % or more of the total fat is obtained from an animal source. Alternatively, about 50 wt. % or more, about 60 wt. % or more, about 70 wt. % or more, about 80 wt. % or more, or about 90 wt. % or more of the total fat may be obtained from a plant source.

The pet food composition includes protein in an amount that may vary. For instance, the pet food composition may include protein in an amount from about 10 to about 40 wt. %, based on the total weight of the pet food composition on a dry matter basis. The protein may be present in an amount from about 10 to about 36 wt. %, about 10 to about 32 wt. %, about 10 to about 28 wt. %, about 10 to about 24 wt. %, about 10 to about 20 wt. %, about 10 to about 18 wt. %; from about 12 to about 40 wt. %, about 12 to about 36 wt. %, about 12 to about 32 wt. %, about 12 to about 28 wt. %, about 12 to about 24 wt. %, about 12 to about 20 wt. %, about 12 to about 18 wt. %; from about 14 to about 40 wt. %, about 14 to about 36 wt. %, about 14 to about 32 wt. %, about 14 to about 28 wt. %, about 14 to about 24 wt. %, about 14 to about 20 wt. %, about 14 to about 18 wt. %; from about 16 to about 40 wt. %, about 16 to about 36 wt. %, about 16 to about 32 wt. %, about 16 to about 28 wt. %, about 16 to about 24 wt. %, about 16 to about 20 wt. %, or about 16 to about 18 wt. %, including ranges or subranges thereof, based on the total weight of the pet food composition on a dry matter basis.

The protein of the pet food composition may comprise one or more amino acids selected from Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, Serine, or a combination of two or more thereof. In some cases, the one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for canines and felines (and humans) include phenylalanine, leucine, methionine, lysine, isoleucine, valine, threonine, tryptophan, histidine, and arginine. In addition, taurine, while technically not an amino acid but a derivative of cysteine, is an essential nutrient for cats.

A portion of the protein in the composition may be predigested (hydrolyzed) protein. For example, the pet food composition may include protein, where about 40 wt. % or more, about 50 wt. % or more, about 60 wt. % or more, about 70 wt. % or more, about 80 wt. % or more, about 90 wt. % or more, about 95 wt. % or more, about 98 wt. % or more, or about 99 wt. % or more of the protein is predigested (hydrolyzed) protein, wherein the weight percentage is based on the total amount of protein in the pet food composition on a dry matter basis. In some embodiments, e.g., when the pet food composition is formulated to promote weight loss, the portion of protein that is predigested (hydrolyzed) protein may be about 60 wt. % or less, about 50 wt. % or less, about 40 wt. % or less, about 30 wt. % or less, about 20 wt. % or less, or about 10 wt. % or less, based on the total amount of protein in the pet food composition on a dry matter basis. In further embodiments, the amount of protein that is predigested (hydrolyzed) protein is about 10 to about 99 wt. %, about 10 to about 95 wt. %, about 10 to about 90 wt. %, about 10 to about 70 wt. %, about 10 to about 50 wt. %, about 10 to about 30 wt. %; about 30 to about 99 wt. %, about 30 to about 95 wt. %, about 30 to about 90 wt. %, about 30 to about 70 wt. %, about 30 to about 50 wt. %; about 50 to about 99 wt. %, about 50 to about 95 wt. %, about 50 to about 90 wt. %, about 50 to about 70 wt. %; or about 70 to about 99 wt. %, about 70 to about 95 wt. %, or about 70 to about 90 wt. %, including ranges and subranges therein, based on the total amount of protein in the pet food composition on a dry matter basis.

Protein may be supplied by any of a variety of sources known by those of ordinary skill in the art including plant sources, animal sources, microbial sources, or a combination of two or more thereof. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs, and intestines. Plant protein may include, for example, soybean protein, cottonseed protein, and peanut protein. Microbial sources may be used to synthesize amino acids (e.g., lysine, threonine, tryptophan, or methionine) or intact protein, such as proteins from sources listed below.

Examples of protein or protein ingredients include egg protein, chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles, and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The pet food composition may further comprise ash. As described herein, ash consists of compounds that are not organic or water, generally produced by combustion of biological materials. The ash may be present in the pet food composition in an amount ranging from about 1 to about 10 wt. %, based on the total weight of the food composition on a dry weight basis, including all amounts and sub-ranges there-between. In some embodiment, the ash may be present in the food composition in an amount ranging from about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the food composition on a dry weight basis.

The amount of carbohydrate, e.g., calculated as NFE, present in the pet food composition may be from an amount up to about 65 wt. %, an amount up to about 60 wt. %, an amount up to about 55 wt. %, an amount up to about 50 wt. %, an amount up to about 45 wt. %, an amount up to about 40 wt. %, an amount up to about 35 wt. %, an amount up to about 30 wt. %, an amount up to about 25 wt. %, an amount up to about 20 wt. %, an amount up to about 15 wt. %, an amount up to about 10 wt. %, an amount up to about 5 wt. %; about 1 to about 65 wt. %, about 1 to about 55 wt. %, about 1 to about 50 wt. %, about 1 to about 45 wt. %, about 1 to about 40 wt. %, about 1 to about 35 wt. %; about 1 to about 30 wt. %, about 1 to about 25 wt. %, about 1 to about 20 wt. %, about 1 to about 15 wt. %, about 1 to about 10 wt. %, about 1 to about 5 wt. %; about 5 to about 65 wt. %, about 5 to about 55 wt. %, about 5 to about 50 wt. %, about 5 to about 45 wt. %, about 5 to about 40 wt. %, about 5 to about 35 wt. %; about 5 to about 30 wt. %, about 5 to about 25 wt. %, about 5 to about 20 wt. %, about 5 to about 15 wt. %; about 10 to about 65 wt. %, about 10 to about 55 wt. %, about 10 to about 50 wt. %, about 10 to about 45 wt. %, about 10 to about 40 wt. %, about 10 to about 35 wt. %; about 10 to about 30 wt. %, about 10 to about 25 wt. %; about 15 to about 65 wt. %, about 15 to about 55 wt. %, about 15 to about 50 wt. %, about 15 to about 45 wt. %, about 15 to about 40 wt. %, about 15 to about 35 wt. %; about 15 to about 30 wt. %; about 20 to about 65 wt. %, about 20 to about 55 wt. %, about 20 to about 50 wt. %, about 20 to about 45 wt. %, about 20 to about 40 wt. %, about 20 to about 35 wt. %; about 25 to about 65 wt. %, about 25 to about 55 wt. %, about 25 to about 50 wt. %, about 25 to about 45 wt. %, about 25 to about 40 wt. %, about 25 to about 35 wt. %; about 30 to about 65 wt. %, about 30 to about 55 wt. %, about 30 to about 50 wt. %, about 30 to about 45 wt. %; about 35 to about 65 wt. %, about 35 to about 55 wt. %, about 35 to about 50 wt. %; about 40 to about 65 wt. %, about 40 to about 55 wt. %, about 45 to about 65 wt. %, about 45 to about 55 wt. %; or about 50 to about 65 wt. %, including ranges and subranges thereof, based on the total weight of the pet composition on a dry matter basis.

The term “carbohydrate” as used herein includes polysaccharides (e.g., starches and dextrins) and sugars (e.g., sucrose, lactose, maltose, glucose, and fructose) that are metabolized for energy when hydrolyzed. Generally, carbohydrate percentage can be calculated as nitrogen free extract (“NFE”), which can be calculated as follows: NFE %=100%−(moisture %)−(protein %)−(fat %)−(ash %)−(crude fiber %). One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, cornstarch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

In certain embodiments, the pet food composition comprises moisture. The moisture may be present at various amounts or concentrations. In one embodiment, moisture may be present in an amount of from about 3 to about 20 wt. %, based on the total weight of the pet food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the pet food composition. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

The pet food compositions disclosed herein may be wet or dry compositions, and the ingredients can be either incorporated into the food composition and/or on the surface of any composition component, such as, for example, by spraying, agglomerating, dusting, or precipitating on the surface. Additionally, the pet food compositions may be formulated and produced to be in various forms and/or consistencies. For instance, the pet food compositions may, for example, be a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles.

The food products may also include components of more than one consistency, for example, wet/canned food, soft, chewy meat-like particles or pieces as well as kibble having an outer coating and an inner “core” component. In some embodiments, the pet food compositions may be in the form of a kibble or food kibble. As used herein, the term “kibble” or “food kibble” refers to a particulate pellet, e.g., like a component of feline or canine feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the pet food composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins, minerals, and flavoring. Other additives can include glucosamine, chondroitin sulfate, vegetable extracts, herbal extracts, etc.

The concentration of such additives in the pet food composition typically can be up to about 5 wt. %, based on the total weight of the pet food composition on a dry matter basis. For example, the additives may be present in an amount from about 0.01 to about 5 wt. %, about 0.01 to about 4 wt. %, about 0.01 to about 4 wt. %, about 0.01 to about 3 wt. %, about 0.01 to about 2 wt. %, about 0.01 to about 1 wt. %; about 0.1 to about 5 wt. %, about 0.1 to about 4 wt. %, about 0.1 to about 4 wt. %, about 0.1 to about 3 wt. %, about 0.1 to about 2 wt. %, about 0.1 to about 1 wt. %; about 1 to about 5 wt. %, about 1 to about 4 wt. %, about 1 to about 4 wt. %, about 1 to about 3 wt. %, about 1 to about 2 wt. %; about 2 to about 5 wt. %, about 2 to about 4 wt. %, about 2 to about 4 wt. %, about 2 to about 3 wt. %; about 3 to about 5 wt. %, about 3 to about 4 wt. %; about 4 to about 5 wt. %, or any range or subrange thereof, based on the total weight of the pet food composition on a dry matter basis. In some embodiments, the concentration of such additives (particularly where such additives are primarily nutritional balancing agents, such as vitamins and minerals) is from about 0 to about 2 wt. %, based on the total weight of the pet food composition on a dry matter basis. The amount of vitamins may include vitamin B. In some embodiments, the concentration of such additives (again, particularly where such additives are primarily nutritional balancing agents) is from about 0 to about 1 wt. %, based on the total weight of the pet food composition on a dry matter basis. Although the list of foregoing additives may be potentially suitable in some embodiments, one or more of the foregoing additives may be excluded from other embodiments of the pet food composition.

In specific embodiments, the pet food compositions and food products are formulated to address specific nutritional differences between species and breeds of animals, as well as one of more of the attributes of the animal. For example, cat and dog foods, e.g., are typically formulated based upon the life stage, age, size, weight, body composition, and breed.

Sources of proteins, carbohydrates, fats, vitamins, minerals, balancing agents, and the like, suitable for inclusion in the pet food compositions, and particularly in the food products to be administered in methods provided herein, may be selected from among those conventional materials known to those of ordinary skill in the art.

The pet food compositions may include, or in some instance exclude, one or more ingredients selected from beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, L-carnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, keratolytics, egg shell membrane, ancient grain, and a combination of two or more thereof.

The pet food composition may be produced by various methods to achieve the desired pet food composition or desired form for the pet food composition. For example, dry food may be baked or extruded, then cut into individual shaped portions, such as kibbles. In some embodiments, the pet food composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The pet food compositions and food products of the present disclosure can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils, water, animal protein, and the like are added combined with the dry materials. The specific formulation, order of addition, combination, and methods and equipment used to combine the various ingredients can be selected from those known in the art. For example, in certain embodiments, the resulting mixture is processed into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a pet food composition, any ingredient generally may be incorporated into the pet food composition during the processing of the formulation, e.g., during and/or after mixing of the other components of the pet food composition. Distribution of these components into the pet food composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In accordance with another aspect of the invention, a method is provided for improving the health of a pet (e.g., by alleviating pruritus and/or chronic itch), the method comprising: increasing the expression of circulating NRP1 and/or the expression of Bcl-2 by feeding a canine a pet food compositions disclosed herein. The method preferably to reduce pruritus by about 25% or more, e.g., assessed about one hour after consumption of the pet food composition by a pet consuming the pet food composition once a day for six consecutive weeks. In some embodiments, the method may be adapted to reduce pruritus for a pet suffering therefrom by about 28% or more, about 31% or more, about 33% or more, about 35% or more, about 38% or more, or about 42% or more, or about 45% or more, e.g., assessed about one hour after consumption of the pet food composition by a pet consuming the pet food composition once a day for six consecutive weeks.

The method may include providing and/or feeding the pet (e.g., canine) the pet food compositions for 1 or more days, preferably 5 or more days, preferably 7 or more days, preferably 10 or more days, preferably 14 or more days, preferably 30 or more days, or preferably 42 or more days. The method may include feeding the pet one time a day, two times a day, three times a day, or in some embodiments four or more times a day.

In accordance with another aspect of the invention, provided is a method for alleviating pruritus and/or chronic itch, the method comprising: adjusting the amount of circulating quercetin to about 2 μg/kg MBW (Metabolic Body weight) or more, e.g., by feeding the pet a pet food composition disclosed herein once a day for about 3 consecutive weeks or more or about 6consecutive weeks or more. The method may adjust the amount of circulating quercetin within the pet to about 3 μg/kg MBW or more, about 4 μg/kg MBW or more, about 5 μg/kg MBW or more, or about 6 μg/kg MBW or more, assessed about one hour after consumption of the pet food composition by a pet consuming the pet food composition once a day for six consecutive weeks. In some cases, the method may increase the circulating quercetin by about 100% or more, about 150% or more, about 200% or more, about 250% or more, about 300% or more, about 350% or more, about 400% or more, about 450% or more, or about 500% or more, assessed about one hour after consumption of the pet food composition by a pet consuming the pet food composition once a day for six consecutive weeks.

The inventors recognized that dermatitis in canines is a chronic condition that is characterized by pruritis and erythema. Without being limited to any particular theories, it is believed that the pathogenesis of canine dermatological conditions are mediated by lymphocytes, eosinophils and mast cells. It is believed that a significant increase in IL5RA as well as a modest increase in CCR3, IL4, and IL5 were seen in canines with dermatological issues when compared to controls. IL5 mediates the maturation, recruitment and activation of eosinophils. IL5RA is expressed on eosinophils as well as committed eosinophil precursers in bone marrow. FCRL2 is a gene mainly expressed in memory B-cells and plasma cells and is also involved in B-cell receptor signaling. The pet food compositions and methods disclosed herein may advantageously effect CCR3, IL4, and IL5 within a pet consuming such pet food compositions. In some embodiments, the pet food compositions are adapted to improves expression of a marker selected from CCR3, IL4, IL5, and a combination thereof in a pet consuming the pet food composition, assessed about one hour after consumption of the pet food composition by a pet consuming the pet food composition once a day for six consecutive weeks. In further embodiments, the method improves expression of a marker selected from CCR3, IL4, IL5, and a combination thereof in a pet consuming the pet food composition, assessed about one hour after consumption of the pet food composition by a pet consuming the pet food composition once a day for six consecutive weeks.

### EXAMPLES

### Example 1

A non-limiting example pet food composition (Example Composition A) was prepared in accordance with aspects of the invention. A comparative pet food composition (Comparative Composition 1) was also prepared. The formulations for Example Composition A and Comparative Composition 1 are provided in Table 1. The macronutrient values of Example Composition A and Comparative Composition 1 are provided in Table 2.

TABLE 1

| | Comparative | Example |
|---|---|---|
| | Composition 1 | Composition 1 |
| Description | (wt. %) | (wt. %) |
| Rice, brown | 47.6 | 30 |
| Rice, brewers | 15 | 26.3 |
| Egg Dried | 9.7 | 12.7 |
| Rice Protein, Conc 80% | 4.9 | 4.9 |
| Soybean Oil and Coconut oil | 6.3 | 6.3 |
| Flax seed whole brown | 3.2 | 2 |
| Beet, pulp | 2.5 | 2.5 |
| Fish oil | 2.3 | 2.3 |
| Lactic acid, blend 84% | 1.2 | 1.2 |
| Dicalcium phosphate and Calcium | 1.6 | 1.6 |
| carbonate | | |
| Buckwheat hull, powder | | 5 |
| Fruit and Vegetable Blend, Silva | 0.5 | |
| Lipoic acid, alpha, 5% | 0.3 | 0.3 |
| Vitamin Premix, Mineral Premix, | 0.44 | 0.44 |
| Antioxidant Premix, and Vitamin E | | |
| Taurine, Choline chloride, and | 0.34 | 0.34 |
| Methionine | | |
| Misc. (PAL, C17007, Natural Flavor, | 4.3 | 4.3 |
| Potassium Chloride, Sodium Chloride) | | |

TABLE 2

| | Comparative | Example |
|---|---|---|
| Nutrients (as fed basis) | Composition 1 | Composition 1 |
| Ash % | 4.3 | 4.2 |
| Moisture % | 8.3 | 8.1 |
| Fat crude % | 16.7 | 17 |
| Protein crude % | 15.9 | 15.8 |
| Fiber crude % | 1.1 | 3.4 |
| Carbohydrate (NFE) % | 53.6 | 51.4 |
| Rutin μg/g | 4 | |
| Quercetin μg/g | 2.4 | 13.5 |
| Calories kcal/kg | 4718 | 4718 |
| Total dietary fiber % | 4.9 | 8.7 |
| Soluble fiber % | <0.2 | 0.9 |
| Insoluble fiber % | 4.9 | 7.8 |
| Soluble/insoluble fiber | 0.04 | 0.12 |
| ratio | | |
| Omega 3 sum % | 1.9 | 1.7 |
| Omega 6 sum % | 4 | 4.3 |
| Omega-3/Omega-6 ratio | 0.47 | 0.4 |

### Example 2

Twenty-six canines were fed Example Composition A and Comparative Composition 1 to evaluate the effects of Example Composition A. The twenty-six canines included thirteen canines with clinically diagnosed dermatological problems and thirteen pair-matched healthy canines by age and gender. All of the canines had ages ranging from 5.8 years to 14.7 years and body weights ranging from 6.6 to 14.1 kg. The canines were of a mixed gender and were neutered or spayed.

The twenty-six canines were separated into a first group and second group with the two groups being matched by age, gender, and phenotype (namely, whether the canine had dermatological problems or was healthy). The twenty-six canines were then fed an adult maintenance food for 28 days as a pre-feed regimen. After the pre-feed regimen, the first group was fed Example Composition A for 42 days while the second group was fed Comparative Composition 1 for 42 days, as a first feeding regimen. After the first feeding regimen, the first group was fed Comparative Composition 1 for 42 days while the second group was fed Example Composition A for 42 days as a second feeding regimen. Blood samples were collected at three time points: end of pre-feed regimen, end of the first feed regimen, and end of the second feed regimen. All canines were fed to maintain their body weight.

The dermatological health and symptoms of the canines were assessed by a veterinarian using a prescribed questionnaire at the end of pre-feed regimen, end of the first feed regimen, and end of the second feed regimen to determine the severity of pruritus, erythema, skin and ear secretions and alopecia only in dermatitis dogs. The veterinarian questionnaire categorical data was then converted into numerical data before calculating the treatment effect on improvement in the percentage of canines alleviating atopic dermatitis symptoms under four categories: pruritus, erythema, alopecia and skin and ear secretions. In each category, there was a set of questions answered by the veterinarian based on his/her assessment of each canine, with the canines being evaluated on the basis of: no symptoms, mild symptoms, moderate symptoms, or severe symptoms.

For gene expression analyses, only eight dogs diagnosed with dermatological problems showed symptoms of mild, moderate, or severe pruritus and erythema symptoms at the initial baseline evaluation. Gene expression count data was normalized with housekeeping gene(s) by using nanostring “nsolver” tool and the resultant normalized data was log transformed before performing statistical analyses. In order to assess the food effect on the gene expression data, mixed model analysis was performed by using treatment (food) as a fixed effect and animal as a random effect by employing JMP Pro v16.0.

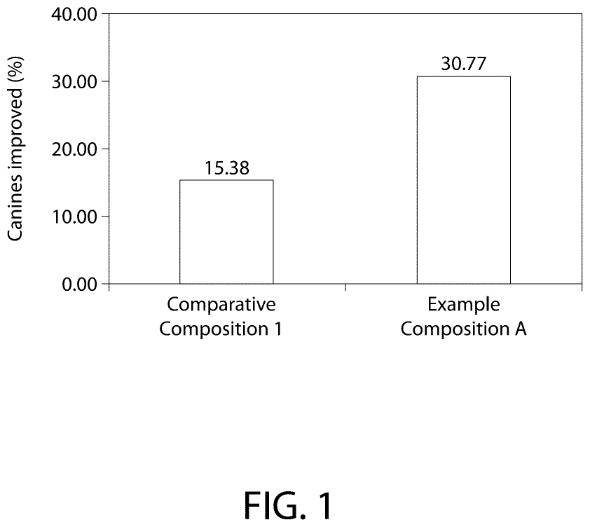

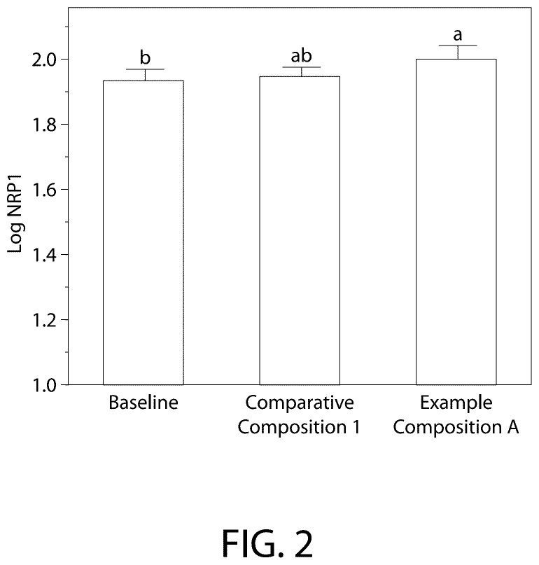

Example Composition A provided a reduction in pruritus scores, e.g., increased the response rate (improvement %), for the canines by about 31%, while Comparative Composition 1 provided a reduction in pruritus scores of about 15% (see FIG. 1). Pruritus is one of the key characteristics of atopic dermatitis symptoms that requires a veterinarian visit, and negatively impacts the pet's quality of life. Based on the gene expression analyses, Example Composition A significantly increased the expression of circulating NRP1 gene when compared to the baseline and also when compared to Comparative Composition 1 (see FIG. 2).

The inventors recognized that pruritus or skin derived itch originates from the skin caused by inflammation, dryness, and/or skin membrane barrier dysfunction, which can be induced by histamine-dependent and histamine-independent signaling pathways. Histamine-dependent signaling pathways are generally responsive to antihistamine agents or drugs that alleviate itch. However, histamine-independent signaling pathways often require non-antihistamine agents or drugs to manage itch and chronic refractory itch is resistant to antihistamine therapies. Without being limited to any particular theory, histamine-independent signaling pathway may involve the NEFs and NRFs, which regulate the hyperinnervation of mechanically sensitive C-nerve fibers. For instance, NRFs, such as Sema3A, may play a role in inhibiting the outgrowth of C-nerve fibers in the epidermal region. Sema3 A forms a complex with the receptor called NRP-1 to induce the signaling pathway to alleviate histamine-independent signaling induced itch. Thus, without being limited to any particular theory, it was surprisingly beneficial that Example Composition A achieved the reduction in pruritus while improving NRP1 gene expression.

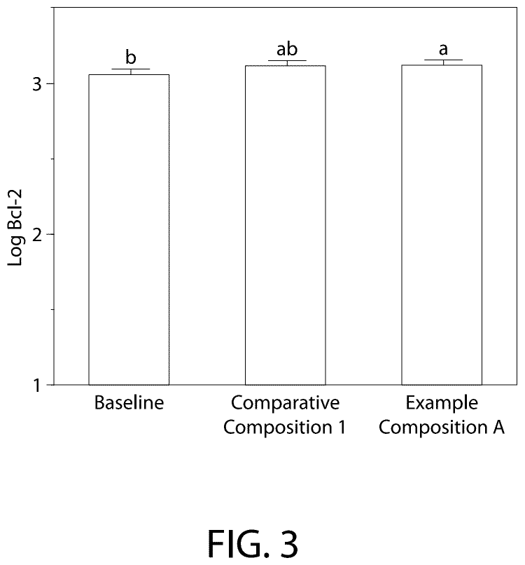

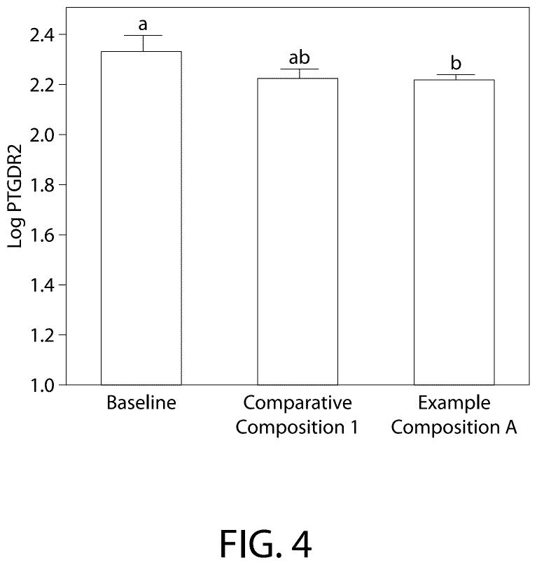

Example Composition A also significantly increased Bcl-2 gene expression when compared to baseline (see FIG. 3). Without being limited to any particular theory, higher levels of Bcl-2 (an antiapoptotic protein) may be associated with epidermal expression of NRP1, which protects keratinocytes from UV-induced apoptosis. In addition, Example Composition A significantly decreased the expression of PTGDR2 (Prostaglandin D2 Receptor 2) compared with the baseline and provided a numerical decrease when compared with the Comparative Composition 1 (see FIG. 4). Without being limited to any particular theory, it is believed that PTGDR2 contributes to allergic skin inflammation upon cutaneous antigen challenge with ovalbumin.

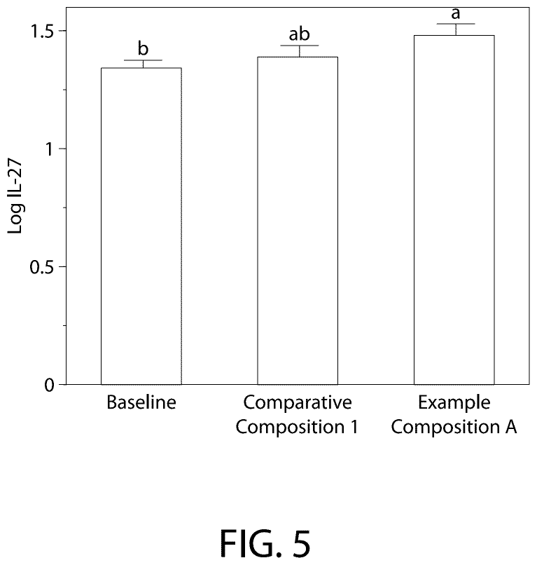

Additionally, Example Composition A significantly increased IL-27 gene expression in dogs fed Example Composition A as compared to the baseline and also a numerical increase when compared with Comparative Composition 1 (see FIG. 5). It is believed that IL-27 facilitates skin wound healing by induction of epidermal proliferation and host defense.

### Example 3

The samples from the eight canines in Example 2 having dermatological problems showing symptoms of mild, moderate, or severe pruritus and erythema symptoms were evaluated. Specifically, the circulating histamine levels in the serum samples from the eight canines were analyzed by using a Histamine Competitive ELISA kit (commercially available from ThermoFisher Scientific) following the manufacturer's instructions.

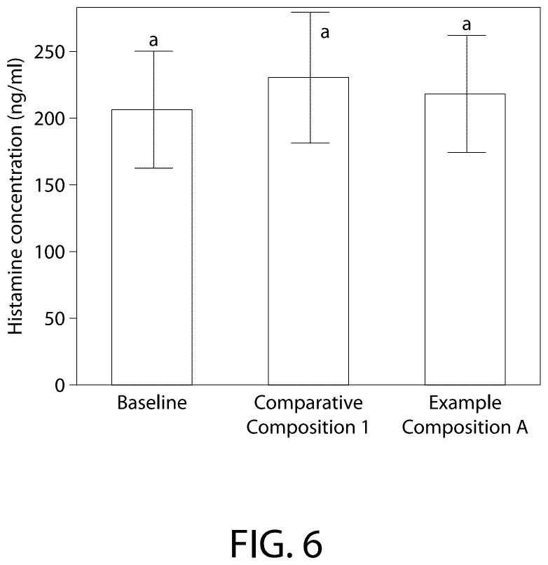

The concentration of histamine was calculated using an eight-point standard curve and reported as ng/ml. To assess the food effect, mixed model analysis was performed by using the pet food composition (either Example Composition A or Comparative Composition 1) as a fixed effect and the animal as a random effect followed by Tukey's post-hoc analysis for multiple comparisons by employing JMP Pro v17.0. Based on the analyses, there were no significant differences in histamine levels between the baseline and Comparative Composition 1 or Example Composition A (see FIG. 6). This data is in agreement with and supports the results from Example 2 that Example Composition A unexpectedly alleviated pruritus and chronic itch mediated by pruriceptors through a histamine-independent signaling pathway (non-histaminergic) in canines with atopic dermatitis. For instance, it is demonstrated that even though clinical signs of dermatitis are reduced at higher levels in Example Composition A compared with Comparative Composition 1, the circulating levels of histamine are similar. This indicates that histamine likely did not mediate a reduction in dermatitis, but that Example Composition A unexpectedly alleviated pruritus and chronic itch mediated by pruriceptors through a histamine-independent signaling pathway.

What is claimed is:

1. A pet food composition comprising:
from about 2 μg/g or more of quercetin;
a fiber component comprising about 8 wt. % or more of a dietary fiber;
a protein; and
a fat,
wherein all weight percentages are based on the total weight of the pet food composition on a dry matter basis.

2. The pet food composition according to claim 1, wherein the dietary fiber comprises a soluble fiber and an insoluble fiber.

3. The pet food composition according to claim 2, wherein the pet food composition comprises a weight ratio of soluble fiber to insoluble fiber of about 1:1 to about 1:15.

4. The pet food composition according to claim 2, wherein the soluble fiber is present in an amount from about 0.1 to about 5 wt. %, the insoluble fiber is present in an amount from about 1 to about 14 wt. %., the protein is present in an amount from about 10 to about 40 wt. %, and the fat is present in an amount from about 9 to about 30 wt. % of fat.

5. The pet food composition according to claim 1 further comprising:
from about 0.1 to about 10 wt. % of a fatty acid.

6. The pet food composition according to claim 5, wherein the fatty acid comprises a polyunsaturated fatty acid.

7. The pet food composition according to claim 6, wherein the polyunsaturated fatty acid comprises an omega-3 fatty acid, an omega-6 fatty acid, or a combination of two or more thereof.

8. The pet food composition according to claim 7, wherein polyunsaturated fatty acid comprises an omega-3 fatty acid selected from linolenic acid, stearidonic acid, eicosatetraenoic acid, eicosapentaenoic acid, docosapentaenoic acid, docosahexaenoic acid, and a combination of two or more thereof.

9. The pet food composition according to claim 7, wherein the polyunsaturated fatty acid comprises an omega-6 fatty acid selected from linolenic acid, calendic acid, eicosadienoic acid, arachidonic acid, docosadienoic acid, adrenic acid, osbond acid, tetracosatetraenoic acid, tetracosapentaenoic acid, and a combination of two or more thereof.

10. The pet food composition according to claim 7, wherein the food composition has a weight ratio of omega-6 fatty acids to omega-3 fatty acids of from about 0.5:1 to about 7:1.

11. The pet food composition according to claim 1, wherein the amount of quercetin is about 2.5 μg/g or more.

12. The pet food composition according to claim 1, wherein the pet food composition is free of rutin.

13. A pet food composition comprising:
from about 2 μg/g or more of quercetin;
from about 8 to about 25 wt. % of a fiber component comprising a plurality of dietary fiber, the plurality of dietary fiber comprising a soluble fiber and an insoluble fiber, wherein the soluble fiber and the insoluble fiber are present in a weight ratio of about 1:1 to about 1:15;
a protein; and
a fat.

14. The pet food composition according to claim 13, wherein the pet food composition is adapted to reduce pruritus by about 25% or more, assessed about one hour after consumption of the pet food composition by a pet consuming the pet food composition once a day for six consecutive weeks.

15. The pet food composition according to claim 13, wherein the pet food composition is adapted to increase expression of circulating NRP1, assessed about one hour after consumption of the pet food composition by a pet consuming the pet food composition once a day for six consecutive weeks.

16. The pet food composition according to claim 13, wherein the pet food composition is adapted to increase expression of Bcl-2, assessed about one hour after consumption of the pet food composition by a pet consuming the pet food composition once a day for six consecutive weeks.

17. The pet food composition according to claim 13, wherein the amount of quercetin is about 2.5 μg/g or more.

18. A method for improving method for alleviating pruritus and/or chronic itch for a canine, the method comprising:
increasing the expression of circulating NRP1 and/or the expression of Bcl-2 by feeding the canine a pet food composition according to claim 13.

19. A method for improving method for alleviating pruritus and/or chronic itch for a canine, the method comprising:
adjusting the amount of circulating quercetin in a pet to about 2 μg/kg MBW or more by feeding the canine a pet food composition according to claim 13.

20. The method according to claim 19 comprising increasing the circulating quercetin in the pet by about 100% or more, assessed about one hour after consumption of the pet food composition by a pet consuming the pet food composition once a day for six consecutive weeks.
